(19) United States
(12) Patent Application Publication
(10) Pub. No.: US 2020/0188121 A1
(43) Pub. Date: Jun. 18, 2020

(54) PATIENT SPECIFIC GLENOID BONE AUGMENT COMPONENTS AND METHODS OF MAKING AND USING THE SAME

(71) Applicant: Tornier, Inc., Bloomington, MN (US)

(72) Inventors: François Boux de Casson, Le Sappey en Chartreuse (FR); Nicolas R. Neichel, Le Sappey-En-Chartreuse (FR); Matthieu Jean Marie Vennin, Grenoble (FR); Willy Vivanz, Cappelle en Pevele (FR); Jean-Emmanuel Cardon, Domene (FR); Delphine Claire Michelle Henry, Saint Ismier (FR)

(21) Appl. No.: 16/785,228

(22) Filed: Feb. 7, 2020

Related U.S. Application Data
(63) Continuation of application No. PCT/US2018/046325, filed on Aug. 10, 2018.
(60) Provisional application No. 62/543,510, filed on Aug. 10, 2017.

(51) Int. Cl. A61F 2/30; A61F 2/40

(52) U.S. Cl. CPC A61F 2/30734; A61F 2/4081; A61F 2002/30985; A61F 2002/4085; A61F 2002/30011; A61F 2002/30952; A61F 2002/30736; A61F 2002/30738; A61F 2002/30784; A61F 2002/30948; A61F 2002/30014

(57) ABSTRACT

A glenoid bone augment component is provided that includes a first side, a second side, and a body that extends between the first side and the second side. The first side is configured to be disposed away from a glenoid of a patient. The second side is configured to be placed on the glenoid. The body is configured to adjust the spacing from the glenoid of a prosthesis component coupled with the first side when the second side of the glenoid bone augment component is coupled with the glenoid. The body has one or both of a central channel and a plurality of peripheral anchor channels disposed therethrough. The channels can be configured to receive a tool for forming an opening in the glenoid. A peripheral reinforcement structure is disposed around at least one of the peripheral anchor channels of the plurality of peripheral anchor channels and/or a central reinforcement structure is disposed around the central channel.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 C.F.R. § 1.57.

### BACKGROUND OF THE INVENTION

### Field of the Invention

This application is directed to patient specific glenoid bone augment components and methods of making and using the same in connection with shoulder arthroplasty and other orthopedic joint surgery.

### Description of the Related Art

Arthroplasty is the standard of care for the treatment of advanced shoulder joint problems, such as severe arthritis. Shoulder arthroplasty can replicate the anatomical form of a joint, with a spherical component mounted on the proximal humerus and a concave surface mounted on the glenoid region of the scapula. Certain patients benefit from a reverse shoulder reconstruction in which a spherical component is mounted to the scapula and a concave surface is positioned on the proximal humerus. Articulation of the spherical component on the concave surface provides the patient with improved arm motion.

One leading reverse shoulder technique, known as bony increased offset reverse shoulder arthroplasty or BIO-RSA provides improved outcomes for patients. BIO-RSA involves placing a spacer between the glenoid region of the scapula and a spherical joint component that is coupled with the glenoid. Among other benefits, BIO-RSA can improve range of motion, limit notching of the scapula, and correct bone deficiency.

Surgeons currently use standard spacers in BIO-RSA, for example with medial and lateral surfaces parallel to each other, disposed at a 12.5 degree angle relative to each other, and with limited options for thickness, such as 7 mm or 10 mm. In cases where the glenoid region surface is worn and may have missing bone portions a spacer with a flat surface will not seat properly against the glenoid and early loosening of the implant can result from improper seating. Further, in cases where the glenoid region surface is worn and may have missing bone portions excessive reaming of the bone may be needed to establish a flat surface on which a spacer can be seated. However excessive reaming of the glenoid surface can remove the dense subchondral bone and expose the soft and porous cancellous bone which is a poor seating surface for the spacer. Early loosening of the implant can be a consequence of excessive reaming. What is needed is a spacer than can conform to the worn glenoid surface without excessive reaming of the scapula.

### SUMMARY OF THE INVENTION

Accordingly, there is a need for improved components for increasing the offset of joint, e.g., glenoid, components from native bone after the bone has been prepared. There also is a need for improved composite ware for such components. There is also a need for improved bone augment components that better suit the needs of patients to provide better securement to the bone of the patient. There is also a need for improved bone augment components that will more fully withstand the compressive loading that arises when prosthetic joints are applied to patients. There is also a need for improved bone augment components that provide enhanced osteointegration properties at or adjacent to bone to which they are coupled and that provide improved mechanical performance at other regions of the bone augment components.

In one embodiment, a glenoid bone augment component is provided that includes a first side and a second side. The first side is configured to be disposed away from a glenoid of a patient. The second side is disposed opposite the first side. The second side is configured to be placed on the glenoid. A body extends between the first side and the second side. The body is configured to adjust the spacing from the glenoid of a prosthesis component coupled with the first side when the second side of the glenoid bone augment component is coupled with the glenoid. The body is disposed around a central channel that extends through the body. The central channel is configured to receive a projection of a portion of a baseplate or a baseplate assembly. The glenoid bone augment component also includes a central reinforcement structure disposed around the central channel.

In certain embodiments, the central reinforcement structure is configured to enhance the strength of the glenoid bone augment component under compression at the central channel.

In another embodiment, a glenoid bone augment component is provided that includes a first side, a second side, and a body that extends between the first side and the second side. The first side is configured to be disposed away from a glenoid of a patient. The second side is disposed opposite the first side. The second side is configured to be placed on the glenoid. The body is configured to adjust the spacing from the glenoid of a prosthesis component coupled with the first side when the second side of the glenoid bone augment component is coupled with the glenoid. The body comprising a plurality of anchor channels disposed therethrough. Tools such as a drill can optionally be advanced through the anchor channels in order to form channels, openings, holes, and/or recesses in the glenoid and/or to otherwise prepare the bone. Peripheral screws or pins can be advanced through the anchor channels to secure the body to a bone segment. A peripheral reinforcement structure is disposed around at least one of the peripheral anchor channels of the plurality of peripheral anchor channels.

In another embodiment a glenoid bone augment component is provided that includes a first side and a second side. The first side is configured to be disposed away from a glenoid of a patient. The second side is disposed opposite the first side. The second side is configured to be placed on the glenoid. The glenoid bone augment component includes a body that has a first region at or adjacent to the first side and a second region at or adjacent to the second side. The body extends between the first side and the second side. The body is configured to adjust the spacing from the glenoid of a prosthesis component coupled with the first side when the second side of the glenoid bone augment component is coupled with the glenoid. The first region comprises a first porosity and the second region comprises a second porosity. The second porosity is greater than the first porosity.

In another embodiment a method is provided in which a medial side of a glenoid bone augment component is placed on a glenoid of a patient. The glenoid bone augment component has a lateral side and a plurality of peripheral anchor, e.g., screw, channels. The peripheral anchor, e.g., screw, channels can be formed at patient specific orientations and/or locations. An articular component is coupled with the glenoid on the lateral side of the glenoid bone augment component. A channel, opening, hole, and/or recess can optionally be formed in the glenoid through each of the channels of the glenoid bone augment component. For example, a tool or instrument can optionally be advanced through each of the channels of the glenoid bone augment component in order to prepare the bone. Each of a plurality of anchors, e.g., screws or pins, is advanced through a corresponding one of the anchor channels of glenoid bone augment component. Each of the plurality of screws is advanced into the glenoid. At least one additional anchor is advanced through a peripheral anchor channel in the glenoid bone augment component into a bone region spaced apart from the glenoid.

The additional anchor can be advanced into the acromion in some methods.

In other aspects of methods the peripheral anchor channels can be formed at non-patient specific orientations and/or locations.

In another embodiment a method of making a glenoid bone augment component is provided. The glenoid bone augment component has a medial surface, a central channel, and at least one peripheral anchor, e.g., screw, channel. The method includes obtaining scapula surface profile information of a specific patient. A manufacturing plan is provided for making the glenoid bone augment component to have a patient specific characteristic in one or more of the medial surface, the central channel, the peripheral anchor channel, a peripheral size, a peripheral shape, and an average thickness. The manufacturing plan is adapted to provide for reinforcement of the glenoid bone augment component around at least one of the central channel and the peripheral anchor channel. The glenoid bone augment component is manufactured according to the manufacturing plan.

### BRIEF DESCRIPTION OF THE DRAWINGS

Various embodiments are depicted in the accompanying drawings for illustrative purposes, and should in no way be interpreted as limiting the scope of the embodiments. Furthermore, various features of different disclosed embodiments can be combined to form additional embodiments, which are part of this disclosure.

FIG. 1 is a partial cross-section of a shoulder joint, including portions of a scapula and a humerus as well as a reverse shoulder joint prosthesis components coupled therewith;

FIG. 2 shows the scapula in cross-section with a baseplate of the glenoid portion of the reverse shoulder joint prosthesis of FIG. 1, the position of the baseplate being spaced from the glenoid by a glenoid bone augment component;

FIG. 3 is a partial cross-section of a scapula having a glenoid component of an anatomical shoulder joint prosthesis coupled therewith;

FIG. 4 is a perspective view of a medial side of another embodiment of a glenoid bone augment component that can be used in reverse and anatomic shoulder joint prostheses, as described herein;

FIG. 5 is a perspective lateral side view of the glenoid bone augment component of FIG. 4;

FIG. 6 is an anterior-posterior cross-section of the glenoid bone augment component of FIG. 4 taken at the section plane 6-6 shown in FIG. 5;

FIG. 7 is a superior-inferior cross-section in perspective of the glenoid bone augment component of FIG. 4 taken at the section plane 7-7 shown in FIG. 5;

FIG. 8A is a schematic view of a scapula showing another embodiment of a glenoid bone augment component and a baseplate coupled with a glenoid of a patient, the glenoid bone augment component also being coupled directly to an acromion of the scapula of a patient; and

FIG. 8B is a lateral side view the glenoid bone augment component and the baseplate coupled with the scapula as shown in FIG. 8A.

FIGS. 9A-9D shows methods of applying the glenoid bone augment and/or bone anchor to a glenoid.

### DETAILED DESCRIPTION OF THE PREFERRED EMBODIMENT

While the present description sets forth specific details of various embodiments, it will be appreciated that the description is illustrative only and should not be construed in any way as limiting. Furthermore, various applications of such embodiments and modifications thereto, which may occur to those who are skilled in the art, are also encompassed by the general concepts described herein. Each and every feature described herein, and each and every combination of two or more of such features, is included within the scope of the present invention provided that the features included in such a combination are not mutually inconsistent.

This application is directed to patient specific glenoid bone augment components and methods for forming and using such components. The glenoid augment components can take several forms as disclosed below, including being configured as grafts made of processed bone matter (autograft, allograft or xenograft) and thus may sometimes be referred to as bone grafts. The glenoid augment components can also be formed of synthetic biologic materials such as a combination of recombinant human platelet derived growth factor BB (rhPDGF-BB) and Beta tri-calcium phosphate (β-TCP) granules. Other materials include bioabsorbable ceramic composite scaffold materials, e.g., that contains 60% hydroxyapatite and 40% β-tricalciumphosphate. Other materials can be configured to maintain space while mimicking natural trabecular bone architecture, e.g., by including 80% BTCP and 20% hydroxyapatite (HA) or 60% BTCP and 40% HA. Bone grafts can also refer more broadly to a component that is configured to enhance a deficiency or fill a gap in bone and such bone grafts can be made of many different materials, including synthetic bone, metals in porous form such as titanium, CoCr, and other porous materials such as Hydroxyapatite (HAP) based materials, tricalcium phosphate (TCP), and ceramics. The glenoid bone augment component can also be a composite of two or more materials. If high porosity is provided in a region of the glenoid bone augment component there can be provided regions comprises harder materials that will not fracture under clinically expected loads. The implant and the graft in various examples can pass various standard test methods like the loosening test method (ASTM2028), the locking mechanism in shear method (ASTM1829), and similar normative test methods.

As discussed above, patient specific glenoid bone augment components can be clinically useful in supplementing eroded, thin or weak bone to which a prosthesis component, such as a glenoid baseplate, is to be coupled. BIO-RSA is one procedure where such augment components can be used. The apparatuses and methods disclosed herein enable novel and less traumatic bone preserving joint replacement techniques. The apparatuses and methods disclosed herein can extend orthopedic treatments to patients who would otherwise not be treatable and can help preserve the possibility of future revisions if needed as well as ensure better connection to bone.

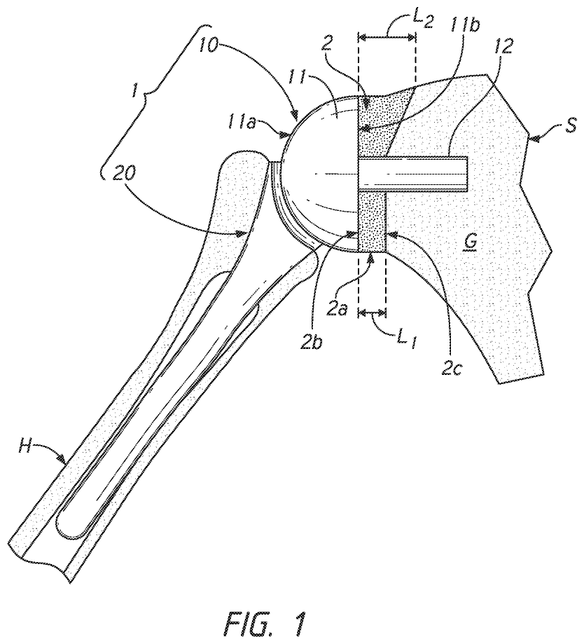

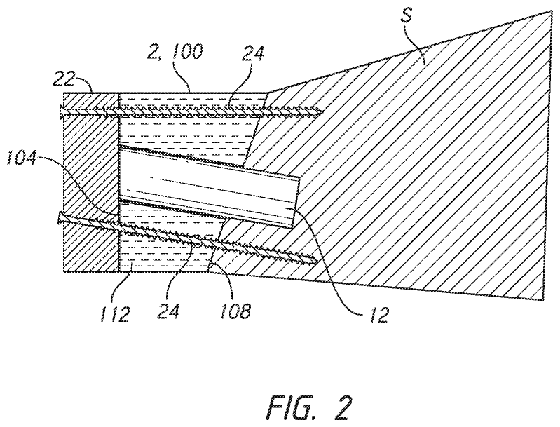

FIG. 1 shows a shoulder prosthesis 1 comprising a glenoid component 10 implanted in the scapula S and a humeral component 20 implanted in the humerus H of a patient's shoulder. The glenoid component 10 has a head 11 which has, on a lateral side, a convex articular surface 11a of generally hemispherical shape. The lateral side is a side facing away from the exposed surface of the glenoid G of the scapula S. The head 11 has an opposing face 11b disposed on a medial side. The medial side is a side facing toward the surface of the glenoid G. In FIG. 1, the face 11b is generally planar but the face 11b can have other shapes. The glenoid component 10 can be configured as a two part assembly with an anchor portion to be secured to the surface of the glenoid and an articular body including the face 11b. FIG. 2 illustrates that the anchor portion can be configured as a baseplate 22 that is anchored to the scapula S with a plurality of peripheral screws 24. Alternatively, as discussed herein, the baseplate 22 can be secured by pins. The baseplate 22 can be further anchored to the scapula S by an anchor member 12 discussed further below, which can be a central post or central screw. Various embodiments of forming the glenoid component 10 as separable components are discussed in U.S. Pat. No. 9,629,725, the entire description thereof being hereby incorporated by reference herein and made a part of this disclosure. FIG. 1 illustrates what is sometimes referred to as a reverse shoulder joint prosthesis because the position of the convex portion of the shoulder joint is the opposite of that of normal anatomy.

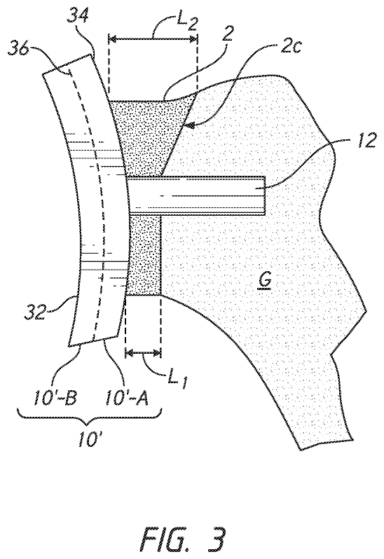

FIG. 3 shows another approach to shoulder replacement where a component 10′ having a concave surface 32 is mounted to the glenoid G. The concave surface 32 is configured to receive a spherical member of a humeral component. An opposing face 34 of the component 10′ is oriented toward the glenoid G. The surface 32 is on a lateral side of and the face 34 is on the medial side of the components 10′. An interface 36 can be present in some embodiments in which the component 10′ comprises an assembly of two separate structures. A first structure 10′-A is configured to be mounted to the surface of the glenoid G in contact therewith and includes the opposing face 34. A second structure 10′-B is coupled with, supported and retained by the first structure 10′-A. The second structure 10′-B includes the surface 32. The interface 36 is illustrated as a dash line because it can be omitted in some embodiments where the component 10′ is not an assembly but rather is a unitary body.

The glenoid components 10, 10′ can also comprise an anchor member 12 that extends from the face 11b (or the face 34) in the direction away from the face 11a (or the surface 32). The free end of the anchor member 12 is securely anchored in the scapula S through the surface of the glenoid G when these components 10, 10′ are implanted to join the components to the scapula S. By way of non-illustrated variation, the anchor member 12 can be externally threaded or, generally, have a surface state promoting bony ingrowth or other mode of anchoring. The anchor member 12 can be separable from or a unitary construction with other the component 10, 10′. Various embodiments of forming the anchor member 12 as a separate component from a baseplate to which a glenosphere comprising the head 1 is mounted are discussed in U.S. Pat. No. 9,629,725, e.g., in connection with FIGS. 3-7E, 81, 8J, 9A-9E, and 10 as well as corresponding text description. These aspects of U.S. Pat. No. 9,629,725 as well as the entire description therein is hereby incorporated by reference into and made a part of this disclosure.

A bone graft 2 is positioned between the surface of the glenoid G and the face 11b, 34 of the respective glenoid components 10, 10′. The bone graft 2 has a periphery 2a and a lateral surface 2b. The lateral surface 2b is located on the side of the bone graft 2 disposed away from the surface of the glenoid G. The lateral surface 2b is medial of the medial face 11b of the head 11 or of a baseplate that is separable from the head 11. The lateral surface 2b can be disposed medial of the face 34 of the component 10′. A medial surface 2c of the bone graft 2 faces and usually is in direct contact with the surface of the glenoid G. Once the bone graft 2 is coupled with the glenoid G, the effective glenoid surface is displaced laterally outward to the distal surface 2b of the bone graft 2.

FIG. 1 shows that the bone graft 2 can have a non-constant thickness. For example, a dimension L₁ at the inferior portion of the glenoid can be smaller than a dimension L₂ at a superior portion. Providing greater medial-lateral thickness at the superior portion can compensate for bone degradation or wear. Because the bone degradation or wear of every patient is different, the best thickness of the bone graft 2 for superior and inferior portions (or for anterior or posterior or other portions) can be different from other portions for each given patient. As such, the bone graft 2 and other glenoid bone augment components discussed herein can be patient specific in various ways in different embodiments discussed below. Specific methods and apparatuses to discern optimal or advantageous thickness profiles, medial surfaces or other patient specific configurations of the bone graft 2 or other augment components and to form the bone graft or other augment components accordingly are discussed hereinbelow. As discussed further below, the medial face 2c preferably has a three-dimensional morphology that matches the three-dimensional morphology of, e.g., is a negative of, the surface of the glenoid G or of the glenoid as the clinician intends to modify it prior to mounting the glenoid component 10, 10′ to the scapula.

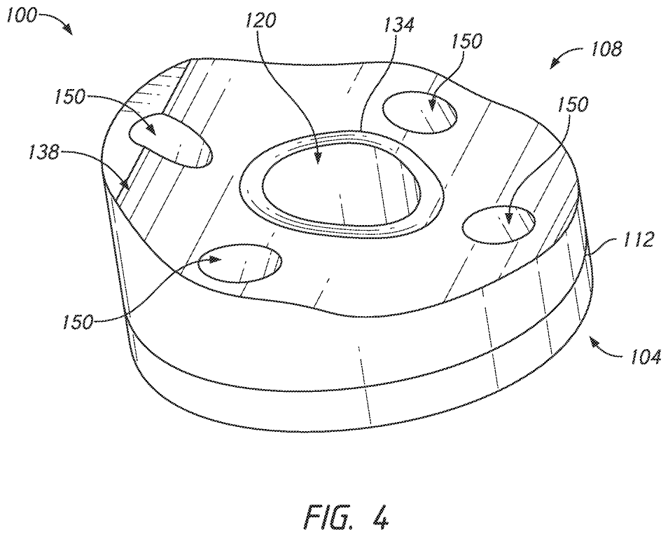

FIG. 4 shows one embodiment of a glenoid bone augment component 100 that is patient specific in any one or more of a plurality of ways as discussed below. The glenoid bone augment component 100 includes a first side 104 and a second side 108. The first side 104 is configured to be disposed away from a glenoid, such as the glenoid G, of a patient. The first side 104 is a lateral side of the glenoid bone augment component 100. The second side 108 is opposite the first side 104. The second side 108 is a medial side of the bone graft 2. The second side 108 is configured to be placed on the glenoid G of the patient. The glenoid bone augment component 100 includes a body 112 that extends between the first side 104 and the second side 108. The body 112 is configured to adjust the spacing from the glenoid G of a prosthesis component, such as the reverse glenoid component 10 or the anatomical component 10′. FIG. 2 shows a baseplate 22 that can be coupled with the medial side of the glenoid component 10, shown in FIG. 1 and with the lateral side of the bone graft 2. Similarly, the baseplate 22 can be coupled with the glenoid augment component 100. When the baseplate 22 is coupled with the first side 104 and the second side 108 is coupled with the glenoid G, the body 112 adjusts the spacing of the baseplate 22 or other aspects of the reverse glenoid component 10 or the baseplate 22 and the anatomic component 10′ coupled therewith from the glenoid G.

The body 112 is disposed round a central channel 120. The central channel 120 is disposed through the body 112 and extends therethrough from a first side 104 to a second side 108. The central channel 120 is configured to receive a portion of, e.g., a medial projection of the baseplate 22 or other prosthesis component. The medial projection can be an integral post or a separable component such as a central screw as discussed above. The projection of the baseplate 22 can be configured as or can be coupled with an anchor to be secured to the bone of the glenoid G. The projection of the baseplate 22 can include a porous surface, threads, barbs, fins or other structures configured to provide or enhance retention of the baseplate 22 in the bone. U.S. Pat. No. 9,629,725 which is incorporated by reference herein above in its entirety, discloses various embodiments of a baseplate 22 that can be secured to the glenoid G by central screws, pegs, or other members.

In some embodiments the central channel 120 can include a central reinforcement structure 134. The central reinforcement structure 134 can be disposed about the central channel 120. The central reinforcement structure 134 can comprise a cylindrical layer or member 200. The layer or member 200 can be a separate component disposed within the body 112. For example, the body 112 can be configured to provide a spacing function to cause the first side 104 to be lateralized. The member 200 can be provided in the local area of the central channel 120 to strengthen the body 112 around the central channel. In some embodiments, the member 200 can be formed as a discrete cylinder disposed between the body 112 and the lumen of the central channel 120. The discrete cylinder can have a thickness discussed below. The member 200 can be formed of a different stronger material than is used for portions of the body 112 that are spaced away from the central channel 120. In one example, the body 112 spaced away from the central channel 120 can comprise a natural or synthetic biological material as discussed above whereas the member 200 can be configured as a tubular layer comprising a stronger material such as a metal such as titanium, cobalt-chromium, or the like or a composite material that can bear much higher compression loads without fracturing or otherwise failing. The body 112 spaced away from the central channel 120 can comprise a material that has enhanced osteointegration properties whereas the member 200 can have a different structure (e.g., a different material or a different material structure of the same material) causing the member 200 to have a higher load bearing capacity. The body 112 spaced away from the channel 120 can comprise a material whereas the member can be a cylindrical member that combines that same material with a second material or structure, e.g., with fibers capable of maintaining the structural integrity of the member 200 under anatomic loading.

The member 200 can be a cylindrical member that has a lesser porosity than that of the portions of the body 112 disposed away from the channel 120. The higher porosity portions of the body 112 can provide greater, stronger and/or faster integration with the glenoid G. The lesser porosity of the member 200 can provide higher strength at least in compression than other portions of the body 112. The porosity of the member 200 can comprise a lower average pore size than at least exposed surface (e.g., medial) portions of the body 112, e.g., a porosity of about 200 microns or less, of about 250 microns or less, or of about 300 microns or less. In some cases, porosity can be provided as a percentage. For example, the porosity of the member 200 can be from 0 to about 45 percent. The porosity of the member 200 can be from 0 to about 20 percent. The porosity of the member 200 can be from 0 to about 10 percent.

The cylindrical member 200 can be disposed along or adjacent to the central channel 120. The cylindrical member 200 can be disposed circumferentially around or surround the central channel 120 in some embodiments. The cylindrical member 200 can be disposed along a length Lc of the central channel 120. The length Lc can extend from the first side 104 to the second side 108 of the glenoid bone augment component 100. The cylindrical member 200 can include a radially projecting flange portion disposed at or adjacent to the first (lateral) side 104 of the component 100. The flange portion can provide additional reinforcement and/or enhanced integration into the radially outward portions of the component 100. The member 200 can have a radial thickness that will depend on factors such as the material selection and porosity thereof, but preferably is at least about 0.5 mm, and be in a range from 0.25 mm to about 6 mm, in some examples between 0.75 mm and 5 mm, in some examples between about 1 and about 4, in various examples between about 1.5 mm and about 1.5 mm. In some examples, the member 200 can have a radial thickness that is not more than about 3 mm.

The central reinforcement structure 134 can comprise an integral or monolithic portion of the body 112 in some embodiments. The structure 134 can strengthen the component 100 by virtue of differentiated material properties. The compressive strength can be made higher in the reinforcement structure 134 than in other portions of the body 112. The compressive strength can be enhanced by reducing the porosity at the surface of or throughout the reinforcement structure 134. The compression strength can be altered by other techniques, such as modifying a molecular structure within the material forming the reinforcement structure 134. As discussed herein, this can be accomplished by modifying the additive manufacturing process in and around the central channel 120.

Additive manufacturing can form the reinforcement structure 134 as a monolithic as with a continuous material with that of the outlying portions of the body 112 but with different material arrangement providing enhanced compressive strength. Additive manufacturing can provide a distinct boundary layer between the member 200 and material of the outlying portions of the body 112, such that stronger materials, composite materials or other strength enhancing configurations can be provided in the member 200.

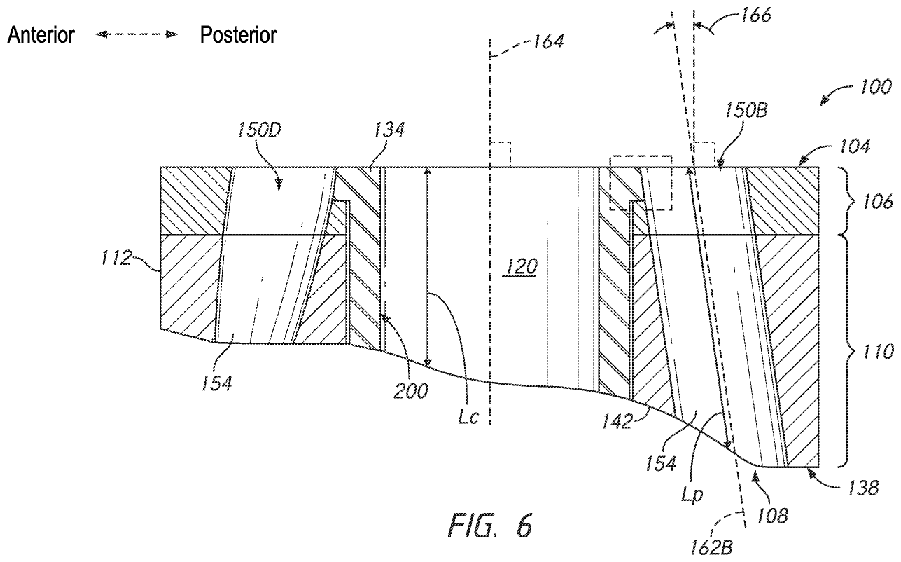

The second side 108 can include a medial surface 138 of the glenoid bone augment component 100. The medial surface 138 can include a patient specific profile 142. The patient specific profile 142 can be seen in an anterior-posterior cross-section of the glenoid bone augment component 100 in some embodiments as shown in FIG. 6. The patient specific profile 142 can be seen in an anterior-posterior cross-section in some embodiments as shown in FIG. 6. The patient specific profile 142 is provided in some embodiments as one example of how the glenoid bone augment component 100 can be made patient specific. The patient specific profile 142 can be formed by obtaining patient images, such as CT scans or MRI images or the like of the glenoid surface of the specific patient to be treated. Thereafter the images are processed using software to determine a relevant bone contour to be matched by the patient specific profile 142. An example of a relevant bone contour is the three dimensional contour of the glenoid G that exists prior to surgery, e.g., the natural contour of the glenoid G. Another example of a relevant bone contour is one that is provided for the specific patient using a patient specific pre-scribed process such as reaming at two or more orientations based on specific patient need. The contour can include flat surfaces disposed at an angle to each other, which surfaces are selected to minimize the extent of reaming for a specific patient. Other patient specific aspects of various embodiments of the glenoid bone augment component 100 are discussed below.

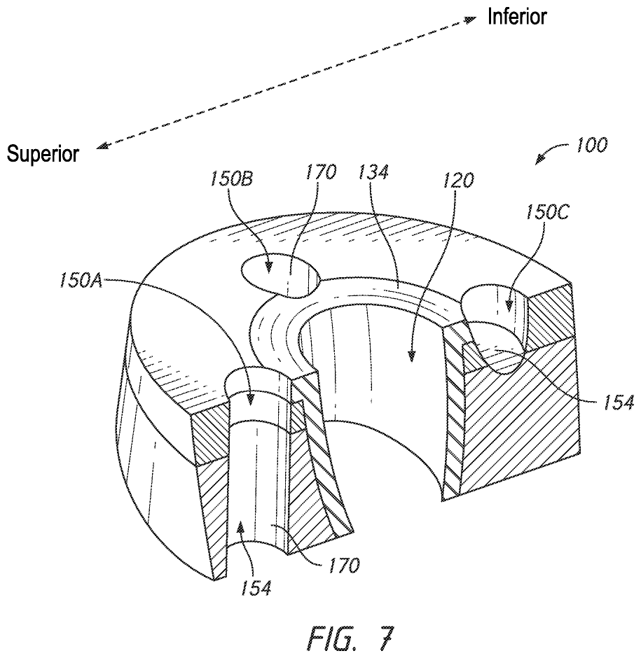

In some embodiments the glenoid bone augment component 100 can be secured to the glenoid G using a one or a plurality of screws, such as the screws 24, placed in a corresponding one or a plurality of peripheral screw channels 150. Although described as screw channels, the channels 150 are examples of anchor channels. In other embodiments, non-threaded anchors such as pins or posts can be advanced through the channels 150 without any threaded engagement therebetween. In some embodiments, some or all of the channels 150 can optionally be configured to receive a tool or instrument for forming a channel, opening, hole, and/or recess in the glenoid. For example, a drill can optionally be advanced through channel 150A, 150B, 150C, and/or 150D in order to prepare the bone. FIG. 7 shows that one of the peripheral screw channels 150 includes a peripheral screw channel 150A disposed at a superior aspect of the glenoid bone augment component 100. The superior peripheral screw channel 150A can be mated to a superior aspect of the glenoid G, as discussed further below. The superior peripheral screw channel 150A can be mated to a portion of the scapula adjacent to a superior aspect of the glenoid G. FIG. 7 also shows that the peripheral screw channels 150 can include a peripheral screw channel 150C disposed at an inferior position on the glenoid bone augment component 100. The patient specific profile 142 can provide in a given patient that the thickness of the body 112 is greater in one of the superior peripheral screw channel 150A and the inferior peripheral screw channel 150C than in the other of the superior peripheral screw channel 150A and the inferior peripheral screw channel 150C.

FIG. 6 shows that the glenoid bone augment component 100 includes a peripheral screw channel 150B disposed in a posterior aspect of the glenoid bone augment component 100. The peripheral screw channel 150B is configured to be mated to a posterior portion of the glenoid G. Also, the glenoid bone augment component 100 can include a peripheral screw channel 150D that is disposed in an anterior portion of the glenoid bone augment component 100. The patient specific profile 142 can provide that a length Lp of the peripheral screw channel 150B is greater than a corresponding length of the peripheral screw channel 150D for a given patient. The configuration illustrated in FIG. 6 corresponds to a patient having a greater amount of erosion or degradation of bone in the posterior aspect of the glenoid G.

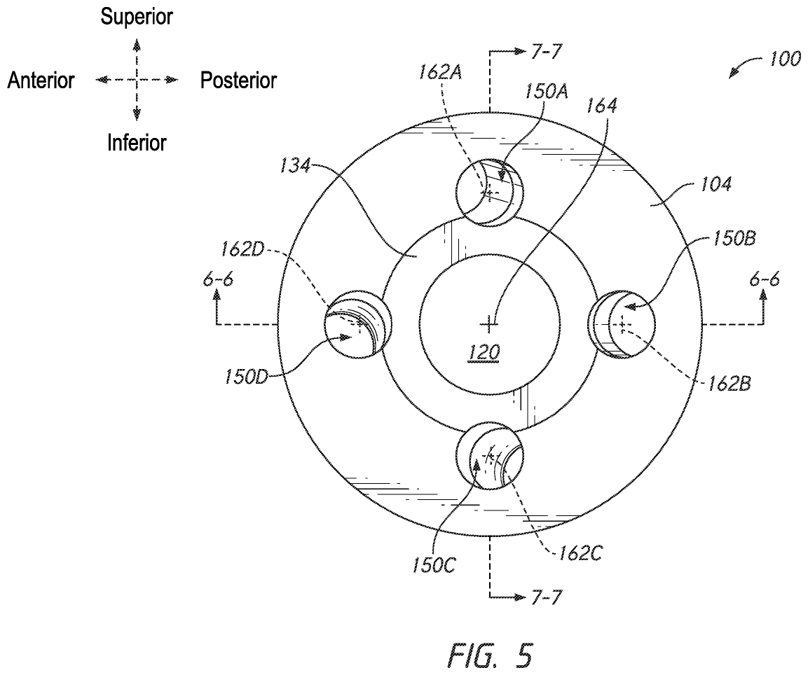

FIGS. 5-7 illustrate another aspect in which the glenoid bone augment component 100 can be made patient specific, e.g., with the peripheral screw channels 150 configured in patient specific ways. FIG. 5 shows that the peripheral screw channels 150 can have orientations that need not be uniformly distributed or oriented. For example, for some patients the peripheral screw channels 150 are not perpendicular to the first (lateral) side 104 of the glenoid bone augment component 100 but rather are oriented according to the specific needs of a given patient. For example, the superior peripheral screw channel 150A can be oriented somewhat anteriorly (or toward the left in FIG. 5). FIG. 5 shows that the lateral side of the superior peripheral screw channel 150A can be centered on the section plane while FIG. 7 shows that the superior peripheral screw channel 150A angles away from the section plane such that the entire medial end of the superior peripheral screw channel 150A is anterior of the section plane. The anterior orientation of the superior peripheral screw channel 150A can reflect that for the given patient the bone stock directly medial of the end of the peripheral screw channels 150A at the first side 104 is less extensive or otherwise less available than the bone mass anteriorly thereof.

The crosshair symbols, as illustrated in FIG. 5, represent the central longitudinal axes 162A, 162B, 162C, 162D, and 164 of the channels 150, 120 of the glenoid bone augment component 100. The axes 162, 164 can extend at non-perpendicular angles to the first or lateral side 104 of the component 100. The first side 104 is the side of the component 100 opposite the second side 108 (the patient-specific side) of the component 100. The axes 162, 164 can extend at inclined angles relative to the first side 104. For example, as illustrated in FIG. 6, the central longitudinal axis 162B of peripheral channel 150B extends at an acute angle 166 relative to an axis that is normal to the first side 104. The axes 162A, 162C, 162D, and 164 can also be positioned at inclined or acute angles.

In some embodiments, the axes 162, 164 can extend at perpendicular angles to the first side 104 of the component 100. For example, as illustrated in FIG. 6, the central longitudinal axis 164 of the central channel 120 extends perpendicular to the first side 104. The inclinations of the axes 162A, 162B, 162C, 162D, and 164 can be dissimilar or similar to one another. The inclinations of the axes can be planned pre-operatively. This can enable an anchor to be advanced into its target location or planned position (e.g. can enable a tip of a screw to be positioned in the cortical bone, without being exposed external to the glenoid, or in another preferred location).

FIG. 5 shows that the peripheral screw channel 150C located inferiorly can be made patient specific. The inferior peripheral screw channel 150C can be directed somewhat posteriorly rather than extending perpendicularly to the first side 104 of the glenoid bone augment component 100. As a result, the medial end of the peripheral screw channel 150C on the second side 108 is shifted posteriorly relative to lateral end of the peripheral screw channel 150C on the first side 104. The length of the peripheral screw channel 150C between the ends thereof extends non-perpendicularly from the first side 104. This orientation can be patient specific for example reflecting a conclusion by the clinician that upon review of patient specific information such as imaging data reveals that the bone stock somewhat posterior is more extensive or more available for securement of peripheral screws than is the bone directly medially of the lateral opening of the peripheral screw channel 150C at the first side 104 of the glenoid bone augment component 100. This can enable a threaded or other anchor portion of a peripheral screw advanced through the peripheral screw channel 150C to engage this more extensive or available bone stock.

Similarly, the peripheral screw channel 150B located posteriorly and the peripheral screw channel 150D located anteriorly can be configured in a patient specific manner. The posterior peripheral screw channel 150B can be oriented posteriorly. This can mean that the medial end of the peripheral screw channel 150B at the second side 108 can be shifted posteriorly relative to the lateral end thereof at the first side 104. The peripheral screw channel 150D can be oriented anteriorly. This can mean that the medial end of the peripheral screw channel 150D can be shifted anteriorly relative to the lateral end thereof at the first side 104. The degree of posterior shift of the medial end of the peripheral screw channel 150B and/or the anterior shift of the medial end of the peripheral screw channel 150D can be determined for a given patient following acquisition of patient specific information. The degree of shift of the medial end of the posterior peripheral screw channel 150B posteriorly and the degree of shift of the medial end of the anterior peripheral screw channel 150D anteriorly can be selected to enable a medial end of a peripheral screw disposed therethrough to reach bone stock that is more extensive and more available for engaging thread or other anchor features thereof.

FIG. 5 shows that the extent of a shift of a medial end of the superior peripheral screw channels 150 can cause the medial end to be located off of the superior-inferior axis of the glenoid bone augment component 100. The other peripheral screw channels 150 can be configured as is the superior peripheral screw channel 150A such that medial ends of the peripheral screw channels 150 can be entirely shifted from the superior-inferior or anterior-posterior axes, even if the lateral ends thereof have centers on these axes. The peripheral screw channels 150 can be configured such that outer periphery of the medial ends thereof are spaced apart from the superior-inferior or anterior-posterior axes, even if the lateral ends thereof are centered on these axes.

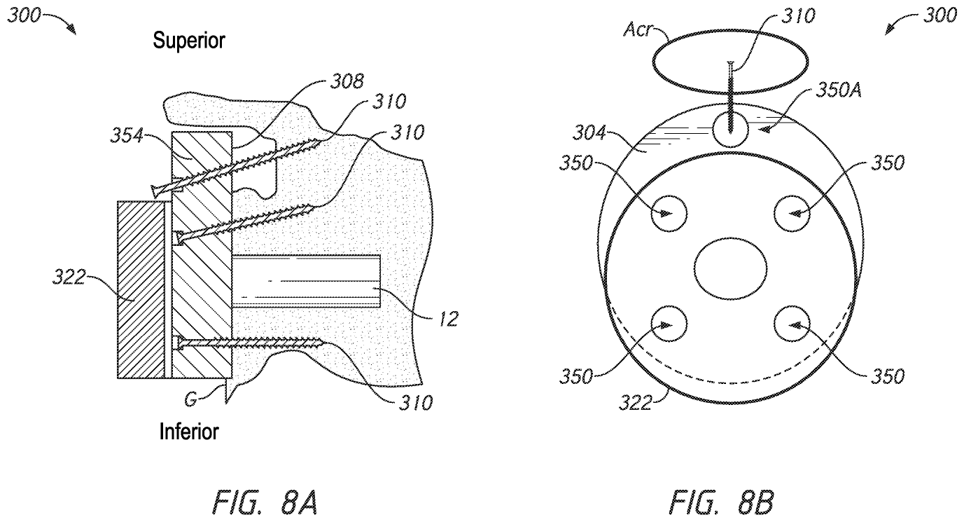

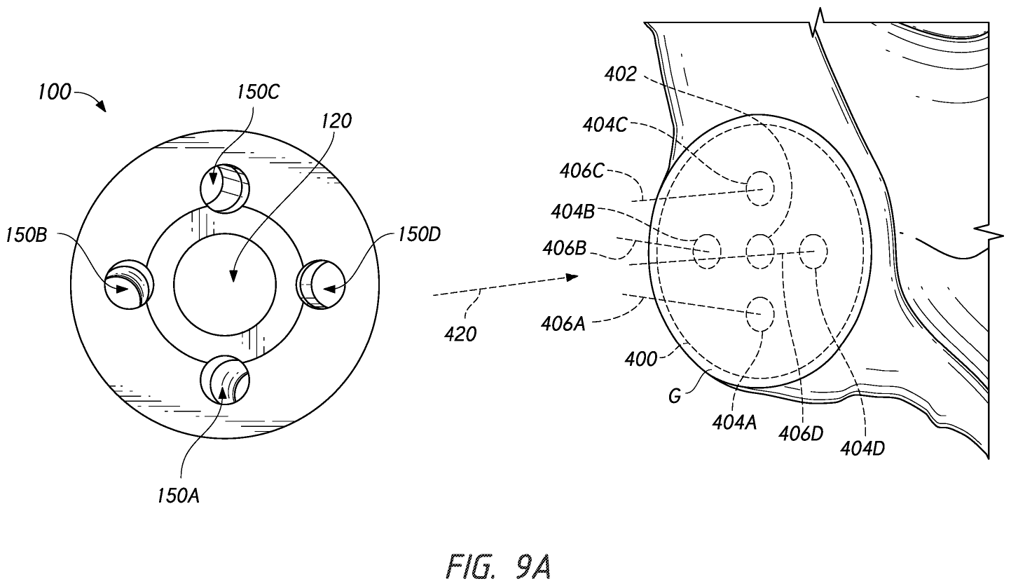

FIG. 5 illustrates that the peripheral screw channels 150 can be disposed at regular non-patient specific locations (such as spaced apart by 90 degrees, at 0°, 90°, 180°, and 270° from the superior position). In other embodiments the locations of one or more of the peripheral screw channels 150 can be patient specific. For example, the superior peripheral screw channel 150A can be located on—that is at 0 degrees relative to a superior-inferior axis. One or more of the other peripheral screw channels 150 can be located away from the superior-inferior axis or away from the anterior-posterior axis. In other embodiments, the superior peripheral screw channel 150A can be generally superior to one or more of the other peripheral screw channels 150 but the center of the peripheral screw channel 150A can be located off of the superior-inferior axis. The inferior peripheral screw channel 150C can be generally inferior to one or more of the other peripheral screw channels 150 but the center of the peripheral screw channel 150C can be located off of the superior-inferior axis. The anterior peripheral screw channel 150D can be generally anteriorly of one or more of the other peripheral screw channels 150 but the center of the peripheral screw channel 150D can be located off of the anterior-posterior axis. The posterior peripheral screw channel 150B can be generally posteriorly of one or more of the other peripheral screw channels 150 but the center of the peripheral screw channel 150B can be located off of the anterior-posterior axis. FIGS. 8(A) and 8(B) show other ways in which the locations and/or orientations of one or more peripheral holes can made patient specific.

As noted above and as shown additional detail in FIGS. 8(A) and 8(B) below, peripheral screws can be used to access through the glenoid bone augment component 100 to provide for anchoring of other structures such as the prosthesis component 11b, the baseplate 22 or other similar structures to bone. When the anchoring is complete the glenoid bone augment component 100 can be compressed between the first side 104 and the second side 108. The compression can be focused around the peripheral screw channels 150 and around the central channel 120 if present. Accordingly, in various embodiments there is an enhancement of the strength of the glenoid bone augment component 100 at one or more of these areas to prevent the augment component from crumbing, degrading or fracturing around these channels.

The peripheral screw channels 150 can be configured with a peripheral reinforcement structure 154. The peripheral reinforcement structure 154 can be disposed around at least one of the peripheral screw channels 150. In some applications one of the peripheral screw channels 150 is planned to receive a screw that generates greater compression and one or more of the peripheral screw channels 150 is planned not to receive a screw producing high compression. The channel receiving the high compression screw can include the peripheral reinforcement structure 154 while the peripheral screw channels 150 planned not receive the high compression screw need not include the peripheral reinforcement structure 154.

The peripheral reinforcement structure 154 can take any suitable form. In the same manner as discussed above in connection with the reinforcement structure 134, the structure 154 can be an integral or monolithic portion of the body 112. Any techniques for enhancing the reinforcement structure 134 can be applied to the reinforcement structure 154. In other embodiments, the peripheral reinforcement structure 154 can comprises a separate cylindrical layer or member 170. The cylindrical member 170 can be disposed along the peripheral screw channels 150. The cylindrical member 170 can have any of the configurations or features discussed above in connection with the member 200. For example, the member 170 can be configured as a cylinder disposed between the body 112 and the lumen of the peripheral screw channel(s) 150. The member 170 can be formed using a different, stronger material than is used for portions of the body 112 that are spaced away from the channels 150. In one example, the body 112 spaced away from the channels 150 can comprise a natural or synthetic biological material as discussed above whereas the member 170 can be a stronger material such as titanium, cobalt-chromium, or other metal or composite that can bear much higher compression loads without failing. The body 112 spaced away from the channels 150 can include a material with enhanced osteointegration properties whereas the member 170 can have a different structure causing the member 170 to have a higher load bearing capacity. The structural difference arise from providing the same material as the body 112 spaced away from the channels 150 but combining that same material with a second material or material structure, e.g., with fibers or other rigid structures capable of maintaining the structural integrity of the member 170 under anatomic loading.

The member 170 can have a lesser porosity than that of at least surface (e.g., medial side) portions of the body 112 disposed away from the channels 150. The higher porosity portions of the body 112 can provide greater, stronger and/or faster integration with the glenoid G. The lesser porosity of the member 170 can provide higher strength at least in compression than other portions of the body 112. The porosity of the member 170 can be can provide an average pore size of about 200 microns or less, of about 250 microns or less, or of about 300 microns or less. In some cases, porosity can be provided as a percentage. For example, the member 170 can be provided with 0 to 45 percent porosity. The member 170 can be provided with 0 to 20 percent porosity. The member 170 can be provided with 0 to 10 percent porosity in some embodiments.

The cylindrical member 170 can extend from the first side 104 to the second side 108 of the glenoid bone augment component 100 in some embodiments. In other embodiments, the cylindrical member 170 can extend from one or both ends of the peripheral screw channels 150 but may not be present along the entire length of the peripheral screw channels 150. In other embodiments, the cylindrical member 170 can be disposed along a central portion of the peripheral screw channels 150 which can be internal to the body 112, e.g. not extending entirely to one or both of the first side 104 and the second side 108. The cylindrical member 170 can surround the peripheral screw channels 150 along a length Lp that extends from at least one of the first side 104 and the second side 108 of the glenoid bone augment component 100. The cylindrical member 170 can have a radial thickness that will depend on factors such as the material selection and porosity thereof, but preferably is at least about 0.5 mm, and be in a range from 0.25 mm to about 6 mm, in some examples between 0.75 mm and 5 mm, in some examples between about 1 mm and about 4 mm, in various examples between about 1.5 mm and about 4.5 mm. In some examples, the member 200 can have a radial thickness that is not more than about 3 mm.

The level of strength enhancement of the reinforcement structures 134, 154 whether configured as a monolithic portion of the body 112 or as separate member or members can be patient specific, e.g., prescribed prior to the procedure based upon pre-operative imaging from CT scans, MRI scans, X-rays or other pre-operative information. For example in some embodiments, the body 112 is formed from a resected portion of the same patient's bone. The humeral head may be resected and thereafter processed into the body 112. If the quality of the bone that is used to form the body 112 is lesser, then a greater level of reinforcement may be provided in one or more of the reinforcement structures 134, 154.

One or both of the reinforcement structures 134, 154 could be configured as temporary structures for reinforcing the component 100. The reinforcement structures 134, 154 could be made of a polymeric material that can degrade or bio-erode over time. For example following osteointegration the component 100 may have lesser or no need for enhanced strength around the channels 120, 150. Thus, the structures 134, 154 may be unnecessary after a period of time. In other applications although the need for reinforcement diminishes or is eliminated, the structures 134, 154 can be made of a permanent polymeric material.

Although the glenoid bone augment component 100 generally is patient specific, it could be configured more generically in some embodiments. When patient specific, the glenoid bone augment component 100 can be formed following acquisition of pre-operative imaging or data describing the actual bone anatomy of the patient to be treated. CT or MRI scan images or X-ray images or the like of the glenoid G, the acromion or other relevant anatomy can be obtained, digitized and analyzed using software. The software is preferably combined with a manufacturing facility that allows the physical structures of the glenoid bone augment component 100 to be made responsive to clinical judgements about the pre-operative images or data. For example, the software can generate a manufacturing plan for making the glenoid bone augment component 100. The plan can include an aspect having a patient specific characteristic in one or more of the medial surface 138, the central channel 120, the peripheral screw channels 150. The plan can include a patient specific peripheral size, a patient specific peripheral shape, and a patient specific average thickness. The peripheral shape of the glenoid bone augment component 100 can include a circular outer periphery. When the shape of the glenoid bone augment component 100 is circular, the diameter can be 20 mm, 25 mm, 29 mm, or other clinically suitable sizes. As discussed in connection with the glenoid bone augment component 300, the shape can be non-circular, such as oval with a major axis aligned with the superior-inferior direction of the component 300.

The manufacturing facility can employ or include additive manufacturing such as three dimensional printing. Examples of three dimensional printing include direct metal laser sintering (DMLS), fused deposition modeling (FDM), fused filament fabrication (FFF), and electron beam melting (EBM). Any one or a combination of these or other additive manufacturing processes can be used to manufacture the augment component 100 or the glenoid bone augment component 300 discussed below or any of the other patient specific devices disclosed herein. In these processes a three dimensional object is formed by sequentially forming individual layers of the object on top of previously formed individual layers. These processes can closely control the gross dimensions of the object and also can form complex features and shapes such as contours. As discussed further below, these processes can be used to form and locate complementary surfaces, such as the patient specific profile 142, on the second (medial) side 108 the glenoid bone augment component 100 to mate with specific anatomy of a specific patient, e.g., a concave profile to nest on top of corresponding convex surfaces. More details of techniques for manufacturing of the patient specific shoulder guide 100 are discussed in WO 2015071757 and WO 2015052586 which are hereby incorporated by reference herein.

The use of additive manufacturing enables the location, trajectory, and strength of the peripheral screw channels 150 to be carefully controlled. For example, the superior peripheral screw channel 150A can be formed by omitting material at the location of the opening thereof in the layer forming the first side 104. The next most medial layer omit material at the same anterior-posterior position or, as discussed above, can be formed by shifting the omitted material a small amount anteriorly. Each subsequent layer can be formed shifting the omitted material slightly anteriorly to provide the superior peripheral screw channel 150A that is non-orthogonal to the first side 104.

The forgoing planning and additive manufacturing processes can be used to produce the reinforcement structure discussed above. For example, a manufacturing plan generated in a method can be adapted to provide or can provide for reinforcement of the glenoid bone augment component 100 around at least one of the central channel 120 and one or more of the peripheral screw channels 150. The user can select which of the peripheral screw channels 150 should be reinforced. The user or the software can determine which materials to use for the body 112 of the glenoid bone augment component 100 and which to use for the reinforcement structure 134. The user or the software can determine what thickness or porosity should be provided in the reinforcement structure 134.

Additive manufacturing also can be used to produce the glenoid bone augment component 100 with a heterogenous porosity at or between the first side 104 and the second side 108. In one embodiment the second side 108 is a medial side of the glenoid bone augment component 100 and is configured to mate directly with the glenoid G. In some techniques it is desired that the glenoid bone augment component 100 foster bony ingrowth from the glenoid G. In some embodiments the porosity of the second side 108 is higher than that of the first side 104. The porosity of the second side 108 is configured to provide enhanced osteointegration. The porosity of the first side 104 can be selected for other performance features. For example, the porosity of the 140 can be lesser than that of the second side 108. The first side 104 can be non-porous in some embodiments.

In one embodiment, the body 112 comprises a first porosity 160 adjacent to the first side 104 and a second porosity 180 at the second side 108. The first porosity 160 is less than the second porosity 180. In other embodiments a first region 106 is provided at or adjacent to the first side 104 and a second region 110 is provided at or adjacent to the second side 108. The body 112 which extends between the first side 104 and the second side 108 can comprise the first region 106 and the second region 110. The first region 106 can comprise the first porosity 160 and the second region 110 can include the second porosity 180. The second porosity 180 is greater than the first porosity 160 to foster greater, stronger and/or faster integration with the glenoid G. In one embodiment the second porosity 180 can provide pores of a size between about 500 microns and about 700 microns. The second porosity 180 can provide in various embodiments an average pore size of about 500 microns, or about 550 microns of about 600 microns, of about 650 microns or of about 700 microns. In various embodiments the first porosity 160 can provide pores of a size between about 200 microns and about 400 microns. In various embodiments, the first porosity 160 can provide an average pore size of about 200 microns, of about 250 microns, or of about 300 microns. In some cases, porosity can be provided as a percentage. For example, 50-80 percent porosity can be provided at the medial surface 138. Lesser porosity, e.g., from 0 to 45 percent porosity, can be provided away from the medial surface 138. Minimal porosity, e.g., from 0 to 20 percent porosity can be provided in areas of stress concentration for example around the central channel 120 or around one or all of the peripheral channels 150. Lower porosity can also be provided at or adjacent to the first side 104. The first side 104 can be smooth with little to no pores. In some variations, the body 112 includes more than two regions such that a smoother transition in porosity can be provided between the second side 108 and the first side 104 if desired. Additive manufacturing is one example of a technology that can enable the porosity of the glenoid bone augment component 100 to vary nearly continuously between the second side 108 and the first side 104 if desired.

The glenoid bone augment component 100 is well suited for coupling with the baseplate 22 to form a portion of a prosthesis, such as the prosthesis 1 or a prosthesis incorporating the anatomic shoulder prosthesis component 10′. With reference to FIG. 2, the glenoid bone augment component 100 can be placed between the baseplate 22 and the scapula S in the place of the bone graft 2.

A method involving the glenoid bone augment component 100 can include forming the glenoid bone augment component 100. The glenoid bone augment component 100 can be formed following first obtaining information characterizing the glenoid G. The glenoid G can be scanned using a CT or MRI scanner. The information characterizing the glenoid G can be input to a system that allows a surgeon to determine various characteristics of the glenoid bone augment component 100. The surgeon can determine features of the glenoid bone augment component 100 to be defined in a subsequent manufacturing process such as the diameter of other size of the periphery 2a of the glenoid bone augment component 100. The surgeon can also determine the location, number, size and/or orientation of the peripheral screw channels 150 that best suits a given patient. In some patients there is insufficient bone stock corresponding to one of the positions shown in FIG. 5. For example if there is insufficient bone stock accessible from the superior position, the surgeon can eliminate the superior peripheral screw channel 150A. The same can be true for any of the other peripheral screw channels 150. As discussed above, the location or orientation of the peripheral screw channels 150 can be altered instead of eliminating them. One or more of the peripheral screw channels 150 can be eliminated where there is sufficient bone stock at one or more other regions even if bone stock is sufficient at the maximum number of positions for the peripheral screw channels 150. This can have the benefit of eliminating zones of stress concentration and of assuring that bone stock is preserved for potential revision procedures.

After the configuration of the glenoid bone augment component 100 is determined the component can be manufactured using additive manufacturing or other suitable processes. The glenoid bone augment component 100 can be made as a separate component from the baseplate 22 or other portion of a glenoid joint component. The glenoid bone augment component 100 can be made as a unitary/integral component with the baseplate 22 or other portion of a glenoid joint component.

After the glenoid bone augment component 100 is formed the component can be mated to the glenoid G. Prior to mating the glenoid bone augment component 100 to the glenoid G the glenoid may be or otherwise modified to be coupled with the component. As discussed above, the patient specific profile 142 can already be suited to mate with the bony surface of the glenoid G so the amount of reaming can be much less than is conventional. This has the benefit of preserving as much of the scapula as possible which can have a number of benefits.

The glenoid bone augment component 100 can be secured to the glenoid G using peripheral anchors, such as the screws 24. The anchors can extend through the baseplate 22 and provide a degree of compression of the glenoid bone augment component 100 to the surface of the glenoid G. Where provided the peripheral reinforcement structure 154 strengthens the structure around the peripheral screw channels 150 to prevent the glenoid bone augment component 100 from fracturing in the areas of stress concentration around the peripheral screw channels 150.

After the glenoid bone augment component 100 is fully secured to the glenoid G by subsequent securement of the baseplate 22 or by simultaneous placement where the glenoid bone augment component 100 includes the baseplate, an articular component can be coupled with the baseplate 22 or the glenoid bone augment component 100.

FIGS. 8A and 8B illustrate another embodiment in which the methods and concepts describe above are employed to provide a patient specific treatment. In a method, a glenoid bone augment component 300 is provided. The glenoid bone augment component 300 can be similar to the glenoid bone augment component 100 and can be planned and made in the same or similar way as the component 100, except as described differently below. The medial side 308 in use is placed on a glenoid G of a given patient for which the glenoid bone augment component 300 was made. The glenoid bone augment component 300 has a plurality of peripheral screw channels 350. The peripheral screw channels 350 can be disposed at irregular positions in various embodiments, e.g. not one at each of 12, 3, 6, and 9 o'clock as in various embodiments of the glenoid bone augment component 100. Any one or more of the peripheral screw channels 350 also can be at non-orthogonal orientations as discussed above.

The glenoid bone augment component 300 can have an exposed peripheral screw channels 350A. The exposed peripheral screw channels 350A can be disposed in an exposed projection 354 of the glenoid bone augment component 300 that is outside of the periphery of an implant baseplate 322 as discussed further below. The exposed peripheral screw channels 350A can be located to provide access for a screw to be directed therethrough without also passing through the implant baseplate 322. FIG. 8(B) illustrates this by showing the outline of the implant baseplate 322 as covering the four inferiorly located peripheral screw channels 350 and not covering the exposed peripheral screw channels 350A, which in this embodiment is disposed superiorly to the superior periphery of the implant baseplate 322.

In other embodiments, the exposed projection 354 could be configured to project in a direction other than superiorly, e.g. could extend anteriorly, posteriorly, or inferiorly.

In one method of using the glenoid bone augment component 300, after the augment components has been coupled with the glenoid G an articular component is coupled with the glenoid G on the lateral side 304 of the glenoid bone augment component 300. The articular component can be a glenosphere an anatomic (concave) articular component. The articular component can either be directly coupled with the glenoid bone augment component 300 or can be indirectly coupled therewith if the implant baseplate 322 is provided as a separate component.

Coupling the implant baseplate 322 with the glenoid G through the glenoid bone augment component 300 can involve placing screws 310 through aligned channels in these structures. As discussed above, the exposed peripheral screw channel 350A can be used to advance a screw 310 to further secure the glenoid bone augment component 300. The exposed peripheral screw channel 350A can be aligned to a portion of the scapula spaced apart from the glenoid G, e.g., into the acromion Acr. Thus, the glenoid bone augment component 300 can be used to secure a glenoid implant to two distinct, spaced apart sections of bone, such as spaced apart sections of the scapula.

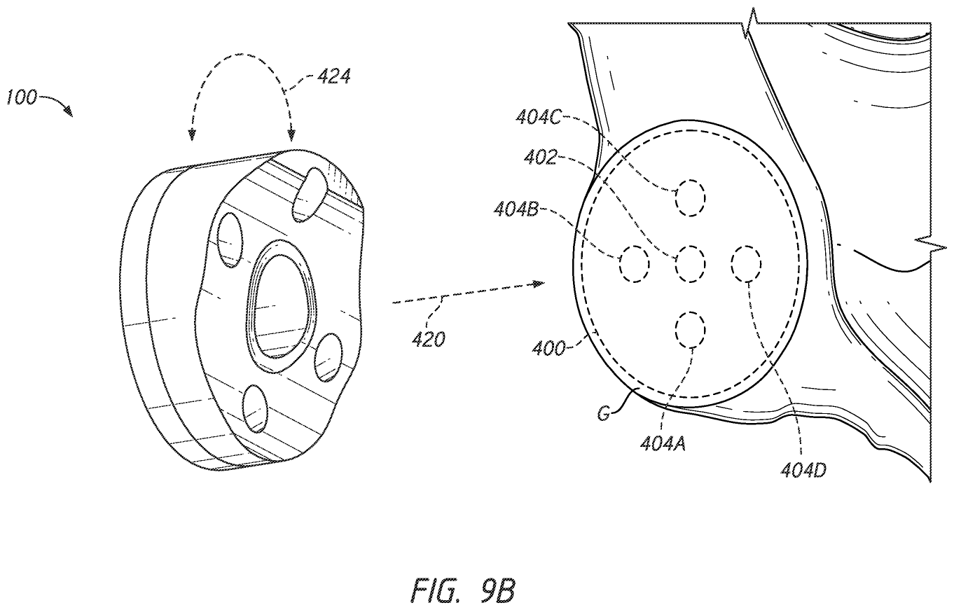

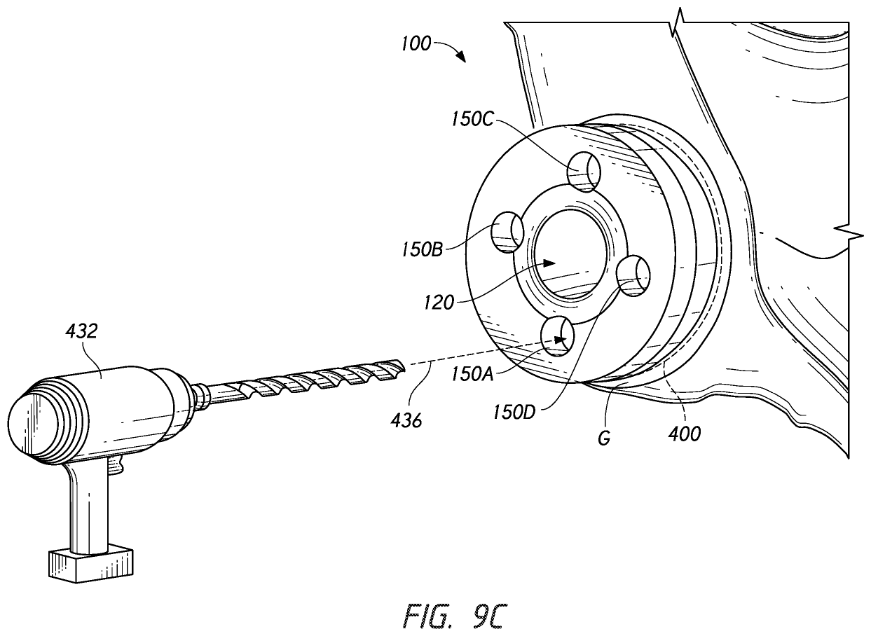

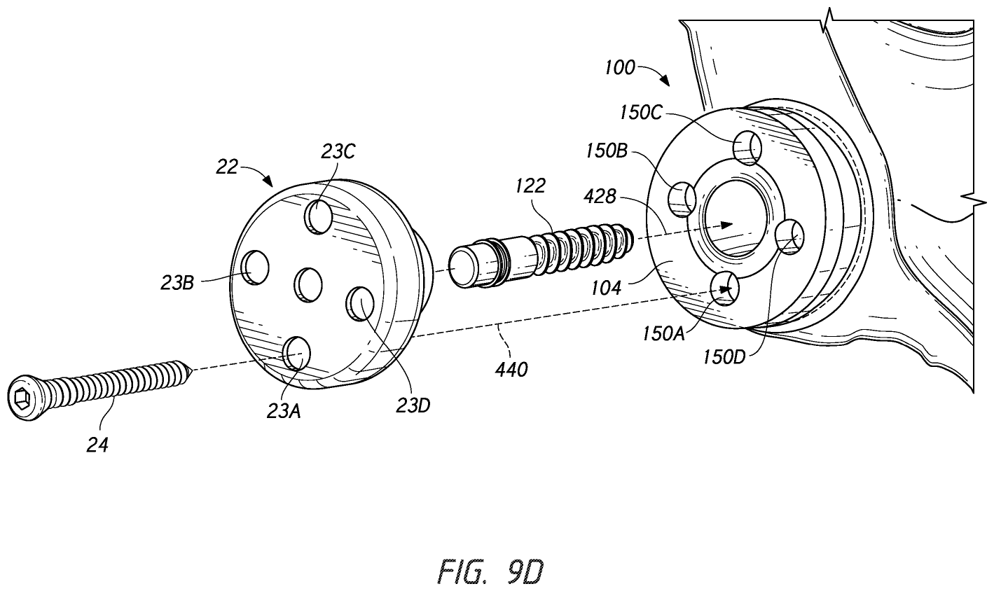

Various methods of planning the application of and also of applying a glenoid bone augment component, such as the component 100 or the component 300 or variations thereof, to the glenoid are discussed in connection with FIGS. 9A-9D. The discussion below is focused on the component 100 but also applies to the component 300 and variants so the discussion is not repeated in its entirety in connection with these variants.

Applying the glenoid bone augment component to the glenoid can include planning or selecting a target location 400 of the glenoid bone augment component 100 on the glenoid G, as illustrated in FIG. 9A. The target location 400 is the region of the glenoid G that will receive and/or support a glenoid component, such as the glenoid bone augment component 100 or the component 300. The positions, sizes, and/or orientations of the channels 150 and/or central channel 120 relative to the desired position of bone anchors can also be pre-operatively planned. Although described as screw channels, the channels 150 can receive anchors of different types and are examples of anchor channels. Non-threaded anchors such as pins or posts can be advanced through the channels 150 without any threaded engagement therebetween.

Software can be used to select the target location 400 and position, size, and/or orientation of the channels of the component 100 relative to the glenoid G. These predetermined locations, sizes, and/or orientations can be selected and/or modified by the user. Peripheral recess entrances 404A, 404B, 404C, and 404D, and central recess entrance 402 represent the predetermined locations of the glenoid that correspond to the channels of the glenoid bone augment component 100. The peripheral recess entrances 404A, 404B, 404C, and 404D are located, e.g., are centered, on locations to be intersected by axes 162A, 162B, 162C, and 162D of the peripheral channels 150A, 150B, 150C, and 150D of the component 100. The central recess entrance 402 is located, e.g., centered on, locations to be intersected by the axis 164 of the central channel 120 of the component 100. The entrances 402, 404A, 404B, 404C, 404D can be displayed on a user interface of a surgical planning tool that can be superimposed on a rendering of the bone of the patient taken from imaging data, e.g., from a CT scan. The entrances 402, 404A, 404B, 404C, 404D identify the locations where recesses can optionally be formed in the glenoid G and/or where anchors can be inserted into the glenoid G.

Peripheral recess entrances 404A, 404B, 404C, and 404D correspond with the respective peripheral channels 150A, 150B, 150C, and 150D. The central recess entrance 402 corresponds with the central channel 120 of the glenoid bone augment component 100. The peripheral recess entrances 404A, 404B, 404C, and 404D align with the respective peripheral channels 150A, 150B, 150C, and 150D when the glenoid bone augment component 100 is positioned on the glenoid G as planned. The axes 406A, 406B, 406C, and 406D of the peripheral recess entrances 404 align with the respective central longitudinal axes 162A, 162B, 162C, and 162D of the peripheral channels 150 when the component 100 is positioned on the glenoid G as planned. The central recess entrance 402 aligns with the central channel 120 of the glenoid bone augment component 100 when the component 100 is positioned on the glenoid G as planned. The axis 408 of the central recess entrance 402 aligns with the central longitudinal axis 164 of the central channel 120 when the component 100 is positioned on the glenoid G as planned.

An arrow 420 indicates a portion of a method in which the glenoid bone augment component 100 is applied to the glenoid G. The glenoid bone augment component 100 can be applied to the target location 400 of the glenoid G such that the peripheral channels 150 and the central channel 120 align with the respective planned peripheral recess entrances 404A, 404B, 404C, 404D and central recess entrance 402. At this point of the procedure these entrances may not have been formed. Applying the glenoid bone augment component 100 to the glenoid G can include placing the component 100 against the glenoid G at the target location 400. The glenoid bone augment component 100 can be oriented in the planned orientation before applying the component 100 to the glenoid G. The component 100 can also be applied to the glenoid G and then re-oriented to obtain the planned orientation.

As shown in FIG. 9B, an arrow 424 indicates that in some methods the user can rotationally orient the glenoid bone augment component 100 until the component 100 is aligned with the glenoid G at the target location 400. The rotation of the component 100 as indicated by the arrow 424 can take place before the glenoid bone augment component 100 contacts the glenoid G, after the component 100 contacts the glenoid G, or both. Rotational orientation of the component 100 as indicated by the arrow 424 can occur prior to step indicated by arrow 420 whereby the glenoid bone augment component 100 is applied to the glenoid G. The patient specific surface of the glenoid bone augment component 100 that engages the glenoid G at the target location 400 enables the component 100 to engage the glenoid G in a particular planned orientation. A portion of the patient specific side 108 of the component 100, opposite side 104, is compatible with the shape of an eroded or worn region of the glenoid G. For this reason, the rotational orientation indicated by the arrow 424 may be performed to align an augmented portion of the component 100 with the eroded or worn region of the glenoid G. In situations where the component 100 has been placed on the glenoid G at the target location 400 and the user rotates the component 100 as indicated by the arrow 424, the component 100 will be biased towards engagement with or held against the glenoid G in the planned orientation in accordance with the shape of the patient specific surface of the component 100.

As illustrated in FIG. 9C, once the glenoid bone augment component 100 is positioned on the glenoid G in the planned orientation, the outer periphery of the component 100 is aligned with the outer periphery of the target location 400. The target location 400 can be displayed on a user interface, such as a screen display of a user interface of a planning system, a heads-up display and other pre-operative or intra-operative systems.

The glenoid bone augment component 100 can optionally be used for forming an opening or recess in the glenoid. Once the component 100 is positioned at the target location 400 of the glenoid G in the planned orientation, the glenoid bone augment component 100 can optionally be used to aid in forming an opening or recess in the glenoid G. In one optional preparation step, a recess extending into the glenoid G from the central recess entrance 402 can be formed through the component 100. For example, as illustrated by an arrow 436, the drill 432 can be coupled with an appropriately sized drill bit that can be advanced through the central channel 120 in the component 100. The surgeon can advance a drill bit coupled with the drill 432 through at least one channel 150 of the glenoid bone augment component 100 (e.g., through channel 150A), and into the glenoid G at the corresponding peripheral recess entrance 404 (e.g., peripheral recess entrance 404A), thereby creating a recess in the glenoid G. The depth and orientation of the recess can be selected pre-operatively and controlled by the length of the bit coupled with the drill 432.

The user can optionally drill recesses extending into the glenoid G from each of the peripheral recess entrances 404A, 404B, 404C, 404D using the glenoid bone augment component 100 as a guide. An axis extends through the center of each of the recesses once the recesses are formed. The axis of each recess is aligned with the center of the corresponding peripheral channel of the glenoid bone augment component 100. Each recess formed in the glenoid G can be similar to or dissimilar from each of the other recesses. One or more of the recesses can extend at a non-perpendicular angle to the first or lateral side 104 of the component 100. Each recess can be configured to have different orientations, e.g., be at different non-perpendicular angles to the first side 104 of the component 100.

In some methods, the baseplate 22 and the anchor member 12 are formed or pre-attached prior to the surgery. The anchor member 12 be an unthreaded post in some embodiments as illustrated above. The anchor member 12 can be advanced through the augment component 100 and further can be advanced into the glenoid surface to be mated to the scapula. As shown in FIG. 2 the outer periphery of the anchor member 12 can be closely matched to the inner periphery of the augment component 100. A recess in the glenoid G can be prepared prior to coupling the augment component 100 and the baseplate 22 with the glenoid or a step of impacting or using peripheral screws to advance the anchor member 12 into the glenoid can be provided.

The arrow 428 in FIG. 9D shows that in some methods a central screw or anchor 122 can be advanced into the central channel 120 of the glenoid augment component 100 into the glenoid G. As discussed above the anchor 122 can be advanced into a pre-drilled hole that is formed using the augment component 100 as a guide. In some methods, the anchor 122 can be directly advanced into the glenoid surface without any preparation of the glenoid surface by drilling. The central screw or anchor 122 can be advanced into an undrilled glenoid area. For example, the anchor 122 can be a self-tapping screw. If the anchor screw 122 is significantly smaller than the inner diameter of the channel 120 an anchor guide can be used to increase the precision of positioning of the anchor 122. For example, a tubular body can optionally be temporarily docked in the central channel 120 to guide the central screw or anchor 122 into an undrilled glenoid area with reference to a pre-planned position and orientation. The central screw or anchor 122 can optionally be advanced into the glenoid after a recess is formed, e.g., drilled, through the central channel 120 in one method.

Then a baseplate 22 is coupled to the component 100. For example, a portion of the baseplate 22 can be coupled to the end of a central anchor or screw 122 opposite the end secured in the glenoid G. The coupling is preferably at the point where the base plate 22 contacts the lateral face of the component 100. The baseplate 22 and central anchor or screw 122 can be unitary or can be separate components that are assembled. The baseplate 22 can have channels that correspond with the channels 120, 150 and the recess entrances 402, 404.

In one variation, once the baseplate 22 is coupled to the glenoid bone augment component 100, an instrument, such as a drill 432, can optionally be used to form holes, channels, or recesses in the glenoid G through the baseplate and the component 100 simultaneously.

As indicated by an arrow 440 as shown in FIG. 9D, a method can include advancing a peripheral anchor, such as a screw 24, through the component 100, and in some cases initially through a channel 23 of the baseplate 22 (e.g. through channel 23A), through the corresponding peripheral channel 150 of the glenoid bone augment component 100 (e.g., through channel 150C), and into the glenoid G. A recess can optionally be formed in the glenoid G prior to advancing the peripheral anchor 24. The peripheral anchor 24 can be advanced into an undrilled glenoid area in one method. For example, the peripheral anchor 24 can be a self-tapping screw. A peripheral anchor 24 can be advanced through each of the channels 23 in the baseplate 22 and/or each of the channels 150 in the component 100, and into the glenoid G. The anchors 24 can secure the baseplate 22 and/or glenoid bone augment component 100 to the glenoid G in a pre-operatively planned manner. The placement of the peripheral anchors 24 can be guided by the augment component 100, which is patient specifically matched to the surface of the pre-operative glenoid G.

Optionally glenoid recesses extending from one or more or all of the entrances 402, 404A, 404B, 404C, and 404D can be formed using a separate guide device that is placed in method steps illustrated by the arrow 420 and optionally by the arrow 424. The guide can be removed from the glenoid G and thereafter the component 100 can be placed in the manner discussed above. This approach may provide the advantage of allowing the component 100 and the baseplate 22 to be pre-assembled.

### Terminology

Although certain embodiments have been described herein with respect to an anatomic component or a reverse component, the implants and methods described herein can interchangeably use any articular component, including the anatomic and reverse components described herein, as the context may dictate.

As used herein, the relative terms “proximal” and “distal” shall be defined from the perspective of the implant. Thus, proximal refers to the direction of the articular component and distal refers to the direction of the base plate when the implant is assembled.

Note that the terms “first” and “second” articular components can be used interchangeably and to refer to the anatomic components or the reverse components. Accordingly, the “first” and “second” openings can be used interchangeably and to refer to any one of the openings in the baseplate.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

The terms “approximately,” “about,” and “substantially” as used herein represent an amount close to the stated amount that still performs a desired function or achieves a desired result. For example, the terms “approximately,” “about,” and “substantially” may refer to an amount that is within less than 10% of the stated amount, as the context may dictate. As an example, in certain embodiments, the term “generally perpendicular” refers to a value, amount, or characteristic that departs from exactly perpendicular by less than about 10 degrees.

Although certain embodiments and examples have been described herein, it will be understood by those skilled in the art that many aspects of the glenoid implants shown and described in the present disclosure may be differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

Some embodiments have been described in connection with the accompanying drawings. However, it should be understood that the figures are not drawn to scale. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Moreover, while illustrative embodiments have been described herein, the scope of any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to the examples described in the present specification or during the prosecution of the application, which examples are to be construed as non-exclusive. Further, the actions of the disclosed processes and methods may be modified in any manner, including by reordering actions and/or inserting additional actions and/or deleting actions. It is intended, therefore, that the specification and examples be considered as illustrative only, with a true scope and spirit being indicated by the claims and their full scope of equivalents.

Any methods disclosed herein need not be performed in the order recited. The methods disclosed herein include certain actions taken by a practitioner; however, they can also include any third-party instruction of those actions, either expressly or by implication. For example, actions such as “inserting a base plate into a glenoid cavity” include “instructing insertion of a base plate into a glenoid cavity.”

1. A glenoid bone augment component comprising:
a first side configured to be disposed away from a glenoid of a patient;
a second side opposite the first side, the second side configured to be placed on the glenoid;
a body extending between the first side and the second side, the body configured to adjust the spacing from the glenoid of a prosthesis component coupled with the first side when the second side of the glenoid bone augment component is coupled with the glenoid, the body disposed around a central channel that extends through the body; and
a central reinforcement structure disposed around the central channel.

2. The glenoid bone augment component of claim 1, wherein the second side comprises a medial surface having a patient specific profile based on a specific patient anatomy as determined by CT scan, MRI scan, or X-ray images or the like.

3. (canceled)

4. The glenoid bone augment component of claim 1, further comprising a plurality of peripheral anchor channels.

5. (canceled)

6. The glenoid bone augment component of claim 4, wherein the peripheral anchor channels are disposed at orientations relative to the first side of the glenoid bone augment component based on a specific patient anatomy.

7. The glenoid bone augment component of claim 4, wherein at least one of the peripheral anchor channels is disposed at locations around the periphery of the body based on a specific patient anatomy.

8. The glenoid bone augment component of claim 4, further comprising a peripheral reinforcement structure disposed around at least one of the peripheral anchor channels of the plurality of peripheral anchor channels, wherein the peripheral reinforcement structure comprises a cylindrical layer or member disposed along the peripheral anchor channel.

9. (canceled)

10. The glenoid bone augment component of claim 8, wherein the cylindrical layer or member surrounds the peripheral anchor channel along a length extending from at least one of the first side of the glenoid bone augment component and the second side of the glenoid bone augment component.

11. The glenoid bone augment component of claim 1, wherein the central reinforcement structure comprises a cylindrical layer or member disposed along the central channel.

12. The glenoid bone augment component of claim 11, wherein the cylindrical layer or member surrounds the central channel along a length thereof extending from the second side of the glenoid bone augment component.

13. (canceled)

14. The glenoid bone augment component of claim 1, wherein the central reinforcement structure is configured to enhance the strength of the glenoid bone augment component under compression at the central channel.

15.-30. (canceled)

31. A method comprising:
placing a medial side of a glenoid bone augment component on a glenoid of a patient, the glenoid bone augment component having a lateral side and a plurality of peripheral anchor channels that are formed at patient specific orientations and/or locations;
coupling an articular component with the glenoid on the lateral side of the glenoid bone augment component;
advancing each of a plurality of screws through a corresponding one of the anchor channels of glenoid bone augment component; and
advancing each of the plurality of screws into the glenoid.

32. The method of claim 31, further comprising forming an opening in the glenoid through each of the anchor channels of the glenoid bone augment component.

33. The method of claim 31, further comprising advancing at least one additional screw through a peripheral anchor channel in the glenoid bone augment component into a portion of a scapula spaced apart from the glenoid.

34. The method of claim 31, further comprising providing a baseplate that is separate from the glenoid bone augment component; placing the baseplate on the lateral side of the glenoid bone augment component; advancing each of the plurality of screws through one of a plurality of channels in the baseplate and through the corresponding one of the anchor channels of the glenoid bone augment component.

35. The method of claim 33, wherein a lateral portion including a lateral end of the at least one additional screw is not disposed through the baseplate.

36. The method of claim 33, wherein the at least one additional screw is advanced superior to the glenoid into the acromion of the patient.

37. A method of making a glenoid bone augment component having a medial surface, a central channel, and at least one peripheral anchor channel, the method comprising:
obtaining scapula surface profile information of a specific patient;
providing a manufacturing plan for making the glenoid bone augment component having a patient specific characteristic in one or more of the medial surface, the central channel, the at least one peripheral anchor channel, a peripheral size, a peripheral shape, and an average thickness;
adapting the manufacturing plan to provide for reinforcement of the glenoid bone augment component around at least one of the central channel and the at least one peripheral anchor channel;
manufacturing the glenoid bone augment component according to the manufacturing plan.

38. The method of claim 37, wherein manufacturing the glenoid bone augment component includes using additive manufacturing to form the glenoid bone augment component.

39. The method of claim 37, further comprising adapting the manufacturing plan to provide a position and/or an orientation of a superior peripheral hole through the glenoid bone augment component that directs a peripheral anchor through the glenoid bone augment component by way of the superior peripheral hole into the acromion when the central channel is centered on the glenoid of the specific patient.

40. The method of claim 37, further comprising adapting the manufacturing plan to provide a porosity at the medial glenoid contact surface that is different than a porosity away from the medial surface.

41.-42. (canceled)

43. The method of claim 37, further comprising finishing the glenoid bone augment component as an augmented baseplate.

44. The glenoid bone augment component of claim 1, wherein the glenoid bone augment component is separable from a baseplate, the glenoid bone augment component enhancing a medial-lateral thickness of the baseplate.

45. The glenoid bone augment component of claim 1, wherein the glenoid bone augment component is integral with a baseplate.
